(19) United States
(12) Patent Application Publication
(10) Pub. No.: US 2017/0266110 A1
(43) Pub. Date: Sep. 21, 2017

(54) DRUG DELIVERY SYSTEM AND METHOD OF TREATING OCULAR DISEASES IN ANIMALS

(71) Applicant: Keith A. Schrunk, Anthon, IA (US)

(72) Inventor: Keith A. Schrunk, Anthon, IA (US)

(73) Assignee: Keith A. Schrunk, Anthon, IA (US)

(21) Appl. No.: 15/076,380

(22) Filed: Mar. 21, 2016

(51) Int. Cl. A61K 9/00; A61K 47/42; A61K 47/46; A61D 7/00

(52) U.S. Cl. CPC A61K 9/0048; A61D 7/00; A61K 47/42; A61K 47/46

(57) ABSTRACT

The present invention relates in general to the field of drug delivery systems for treating ocular diseases in animals, and more specifically, to a drug delivery system and method of treating Infectious Bovine Keratoconjunctivitis (“IBK”) in cattle, commonly known as “pinkeye.” The drug delivery system and method may include a contact lens that has been infused with drugs for treating IBK, such as oxytetracycline, penicillin, streptomycin, tetracycline, gentamicin, cloxacillin or combinations thereof. The medicated contact lens may be placed in contact with the cornea and/or conjunctiva of the afflicted eye of the animal. Drugs are released from the medicated contact lens via diffusion into the cornea and/or conjunctiva of the eye. After treatment, the contact lens may harmlessly dissolve in the eye wherein it is washed away via tear secretions.

### FIELD OF THE INVENTION

The present invention relates in general to the field of drug delivery systems for treating ocular diseases in animals, and more specifically, to a drug delivery system and method of treating Infectious Bovine Keratoconjunctivitis in cattle, commonly known as “pinkeye.” The purpose of the invention is to provide a drug delivery system and method of treating Infectious Bovine Keratoconjunctivitis that utilizes a therapeutic contact lens that dissolves in vivo. An additional purpose of the invention is to provide a drug delivery system and method that effectively reduces dosing frequency and the resultant stress on an afflicted animal during treatment.

### BACKGROUND OF THE INVENTION

“Don't touch your eyes!” a mother instructs her hapless child unlucky enough to contract conjunctivitis, also known as “pinkeye.” Nevertheless, obeying such an order becomes quite difficult as symptoms include itchy eyes, burning sensations, and blurred vision. While the risk of complications during recovery is extremely low for humans, our counterparts in the animal kingdom are not as fortunate. In particular, Infectious Bovine Keratoconjunctivitis (“IBK”) or “pinkeye” is a highly contagious ocular disease afflicting cattle worldwide that is caused by the Gram negative bacterium Moraxella bovis (“M. bovis”). M. Bovis latches onto the surface of the cornea with a hairlike appendage called “pili.” Once attached, the bacterium releases a toxin that kills the cells on the surface of the cornea, leading to ulcers, corneal rupture and permanent blindness in the most severe cases. Able to spread rapidly throughout the herd, IBK causes tremendous losses in weight gain and appetite amongst livestock due to visual impairment and excessive ocular pain. For example, calves afflicted with IBK are on average 35-40 pounds lighter at weaning compared to healthy calves. Post-weaned livestock have stunted average daily gains and final market weights. Dairy animals may also experience up to 25% reduction in milk production. Animals with visual impairments as a result of IBK are at higher risks of fatality through accidents or starvation if they are unable to locate feed and/or water sources. Thus, IBK is a costly disease for beef producers, wherein decreased market values, loss of breeding stock and show animals, reduced milk production, and treatment costs are estimated to be over $150 million annually in the United States alone. According to reports from the National Animal Health Monitoring System of the United States Department of Agriculture, pinkeye is the leading disease affecting all breeding beef females (replacement heifers and cows), and the second most common disease of nursing calves greater than three weeks old. It cannot be overstated that IBK has a staggering negative impact upon the beef industry.

IBK may be transmitted rapidly throughout the herd via a multitude of factors. Transmission initially occurs when a non-infected host comes into contact with secretions (e.g., nasal and ocular discharges) contaminated with the bacterium M. bovis. Any underlying eye irritation allows the bacterium to invade the cornea of the eye and cause pinkeye. Eye irritants include face flies (Musca autumnalis) that feed on secretions around the eyes and nostrils of cattle. Eye irritants also include windblown dust, pollen and seed heads, grazing on tall vegetation, and thorns or barbed wire. Contributing factors such as the particular breed of cattle, age and sex of the animal, nutritional imbalances, and outside stressors may predispose the animal to IBK infection.

IBK was first reported in 1889. More than a century later, control and treatment programs are only partially successful, extremely time-consuming and quite costly. Indeed, the single most important factor in controlling an outbreak of pinkeye throughout a herd is isolating the affected animals and utilizing a fly knockdown spray to reduce new pinkeye cases. With regard to treatment, antibiotics may be delivered to the infected eye via topical application or subconjunctival injection, although such methods have significant disadvantages. Topical antibiotics (e.g., aerosol sprays, ointments, gels, or powders) often further exacerbate infected, irritated eyes. As a result, topical antibiotics rarely remain in the eye long enough to be effective before being quickly washed away via tear secretions. Repeat dosing three to four times daily is thus required to sustain adequate drug levels in the eye. Subconjunctival injection of antibiotics such as penicillin, streptomycin, tetracycline, or gentamycin under the lining of the affected eyelids is recommended where repeated topical treatments are impractical. Intramuscular injections are generally not recommended due to the very high doses of antibiotic required to reach the eyes and tear glands of the animal. Vaccines are available but the results have been disappointing, as there are over twenty different strains of M. bovis and continuous mutations occur in the bacterium. Most pinkeye vaccines require multiple doses and it usually takes at least one month for immunity protection to develop. In extreme cases of pinkeye, an eyepatch or tarsorrhaphy may be utilized, wherein the eyelids are partially sewn together to protect the eye from light, flies and other irritants. Unfortunately, these labor intensive, expensive, and time consuming methods of treating IBK in cattle are not always successful in saving the animal's vision.

Thus, a desire remains to develop a drug delivery system and method of treating ocular diseases in animals that keeps the drug in constant contact with the infected eye without being washed away by tear secretions. A desire also remains to develop a drug delivery system and method of treating ocular diseases in animals that conveniently reduces the need for repeat dosing. A further desire remains to develop a drug delivery system and method of treating ocular diseases in animals that protects the infected eye from foreign irritants and promotes corneal healing.

### BRIEF SUMMARY OF THE INVENTION

Therefore, it is a principal object, feature, and/or advantage of the present invention to overcome the aforementioned deficiencies in the art and provide a drug delivery system and method for treating ocular diseases in animals.

Another object, feature, and/or advantage of the present invention is to provide a drug delivery system and method of treating ocular diseases in animals that is compatible with all species, breeds, sizes and ages.

Yet another object, feature, and/or advantage of the present invention is to provide a drug delivery system and method of treating ocular diseases in animals that is convenient and easy to use by handlers/veterinarians of all ages and physical capabilities.

A further object, feature, and/or advantage of the present invention is to provide a drug delivery system and method of treating ocular diseases in animals that is effective with a single dosage.

A still further object, feature, and/or advantage of the present invention is to provide a drug delivery system and method that treats IBK in cattle.

Another object, feature, and/or advantage of the present invention is to provide a drug delivery system and method of treating IBK that utilizes a therapeutic contact lens.

Yet another object, feature, and/or advantage of the present invention is to provide a drug delivery system and method of treating IBK that utilizes a therapeutic contact lens that dissolves in vivo.

A further object, feature, and/or advantage of the present invention is to provide a drug delivery system and method of treating IBK that reduces stress on both the afflicted animal and handler/veterinarian during treatment.

A still further object, feature, and/or advantage of the present invention is to provide a drug delivery system and method of treating IBK that keeps the antibiotic in constant contact with the afflicted eye without being washed away by tear secretions.

Another object, feature, and/or advantage of the present invention is to provide a drug delivery system and method of treating IBK that conveniently reduces the need for repeat dosing.

Yet another object, feature, and/or advantage of the present invention is to provide a drug delivery system and method of treating IBK that protects the afflicted eye from foreign irritants and promotes corneal healing.

These and/or other objects, features, and/or advantages of the present invention will be apparent to those skilled in the art. The present invention is not to be limited to or by these objects, features, and advantages. No single aspect need provide each and every object, feature, or advantage.

According to one aspect of the present invention, a drug delivery system for treating ocular diseases in animals includes a therapeutic contact lens comprising collagen, amniotic membranes, and/or hydrogels. The therapeutic contact lens may further comprise a therapeutic drug(s). In particular regard to treating IBK in cattle, the therapeutic drug(s) may comprise oxytetracycline, penicillin, streptomycin, tetracycline, gentamycin, Cloxicillin or combinations thereof. The medicated therapeutic contact lens may be placed in the afflicted eye of an animal with the ocular disease. Specifically, the medicated therapeutic contact lens may be placed in contact with the cornea and/or conjunctiva of the afflicted eye. The therapeutic drug(s) are released from the medicated therapeutic contact lens via diffusion into the cornea and/or conjunctiva of the eye. After treatment, the therapeutic contact lens may be biodegradable wherein the lens harmlessly dissolves in the eye and is washed away via tear secretions.

According to another aspect of the present invention a method of treating ocular diseases in animals is provided. The method includes providing an animal with an ocular disease, particularly, IBK in cattle. The method further comprises providing a drug delivery system for treating ocular diseases, wherein the drug delivery system includes a therapeutic contact lens. The method further comprises incorporating therapeutic drug(s) into the therapeutic contact lens. With regard to treating IBK in cattle, the therapeutic drug(s) may comprise oxytetracycline, penicillin, streptomycin, tetracycline, gentamycin, Cloxicillin or combinations thereof. The method of the present invention further comprises placing the medicated therapeutic contact lens in the afflicted eye of an animal. In particular, the medicated therapeutic contact lens may be placed in contact with the cornea and/or conjunctiva of an afflicted eye for a localized application. The therapeutic drug(s) may then be released from the medicated therapeutic contact lens via diffusion into the cornea and/or conjunctiva of the eye. The method of the present invention may also provide ocular surface protection of the eye during treatment. After treatment, the therapeutic contact lens may dissolve in the eye wherein it harmlessly biodegrades and is washed away from the eye via tear secretions. The foregoing steps of the method of the present invention may be repeated if necessary until the ocular disease is successfully treated.

Different aspects may meet different objects of the invention. Other objectives and advantages of this invention will be more apparent in the following detailed description taken in conjunction with the figures. The present invention is not to be limited by or to these objects or aspects.

### DESCRIPTION OF FIGURES

FIGS. 1-4 represent examples of a drug delivery system of the present invention for treating ocular diseases in animals, and a method of treating ocular diseases in animals using the drug delivery system of the present invention.

FIG. 1 is a side view of the parts of the eye in animals.

FIG. 2 is a diagram of a Pinkeye Triad in animals.

FIG. 3 is a view of a biodegradable, therapeutic contact lens for treating ocular diseases in animals.

FIG. 4 is flow-chart of a method of treating ocular diseases in animals.

### DETAILED DESCRIPTION OF THE INVENTION

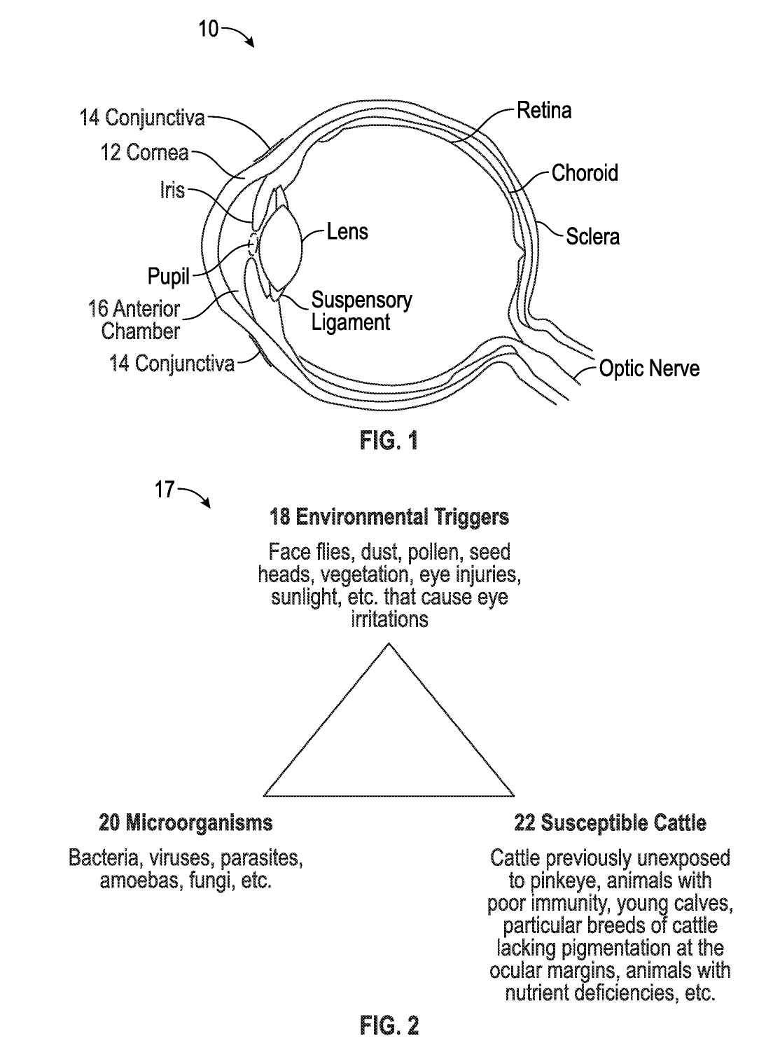

FIG. 1 illustrates a side view of the parts of the eye (10) in animals. As shown in FIG. 1, ocular diseases may originate in animals after eye irritants, such as dust, pollen, face flies, or grasses, scratch the cornea (12) and/or conjunctiva (14) of the eye (10). Once irritated, microorganisms (e.g., bacteria, viruses, parasites, amoebas, fungi, etc.) invade the cornea (12) and/or conjunctiva (14) to cause an ocular disease. “Ocular diseases” in animals may include conjunctivitis (i.e., inflammation of the conjunctiva), keratitis (i.e., inflammation of the cornea), and/or keratoconjunctivitis (i.e., inflammation of the cornea and conjunctiva). Commonly known as “pinkeye,” these ocular diseases may be caused by the aforementioned microorganisms and/or allergens and affect a wide variety of animals. For example, IBK affects cattle and is primarily caused by the bacteria M. bovis. The incubation period for IBK is usually two to three days, wherein redness, swelling and inflammation of the conjunctiva (14), excessive tearing and squinting are the initial clinical signs. In about two days, a small opaque area may appear in the center of the cornea (12) of the eye (10). By the sixth day the entire cornea (12) may have a gray-white to yellowish discoloration with deep central ulceration of the cornea (12). Ulceration may appear as a hole or depression in the cornea (12). At this stage animals have a decreased appetite due to excessive ocular pain, and may have a moderate elevation of body temperature. If left untreated, severe ulceration, cone-shaped bulging of the eye (10), and corneal rupture with loss of eye contents from the anterior chamber (18) may result leading to blindness. With appropriate treatment, recovery may occur within three to five weeks. Afflicted eyes (10) may be left with a permanent white scar on the cornea (12), which can contribute to partial or complete blindness in the eye (10).

FIG. 2 illustrates a Pinkeye Triad (17) demonstrating how ocular diseases are rapidly spread throughout an animal population (e.g., IBK in a cattle herd). As shown in FIG. 2, environmental triggers (18) cause eye irritation, corneal and conjunctival abrasions, and excessive tear secretions. Underlying eye irritations allow microorganisms such as bacteria, viruses, parasites, amoebas, fungi, etc. (e.g., M. bovi) (20) to invade the cornea and/or conjunctivitis of the eye. Environmental triggers (18) that create eye irritations include face flies that feed on secretions around the eyes and nostrils of cattle. Additional environmental triggers (18) include windblown dust particles, pollen and seed heads, grazing on tall vegetation, and eye injuries due to thorns or barbed wire. Cattle eating from overhead feed bunks or from the center of round bales further increase the incidence of eye irritation. Contributing environmental triggers (18) include exposure to excessive UV sunlight which causes damage to corneal epithelial cells. As shown in FIG. 2, the bacteria M. bovis (20) may lay dormant in cattle that exhibit no outward signs of IBK. Indeed, even after an animal has fully recovered from pinkeye the bacteria (20) may remain present in nasal cavities, conjunctiva, and oral secretions. As a result, these carriers allow IBK to remain in the herd and expose susceptible cattle (22) year after year. Carriers, along with animals showing clinical signs of the disease, serve to infect susceptible cattle (22). Transmission may occur rapidly throughout the herd via contact with infected secretions. Face flies are the primary vector for spreading IBK to susceptible cattle (22) since the bacterium (20) may survive on flies' wings and legs for up to four days. Susceptible cattle (22) may comprise particular breeds of cattle (e.g., Hereford, Charolais, and some Holsteins) that lack pigmentation at the ocular margins and are therefore more likely to develop pinkeye because of their increased sensitivity to sunlight and decreased immune response in the eye. Young calves are more susceptible to IBK then adult cattle, and a higher prevalence has been reported in bull calves as compared to heifers. As typical with many diseases, stress caused by weather, transport, weaning, nutritional imbalances, deficiencies of protein, vitamins, and minerals, and the presence of other microorganisms tend to decrease an animal's immune response and predispose the animal to IBK infection.

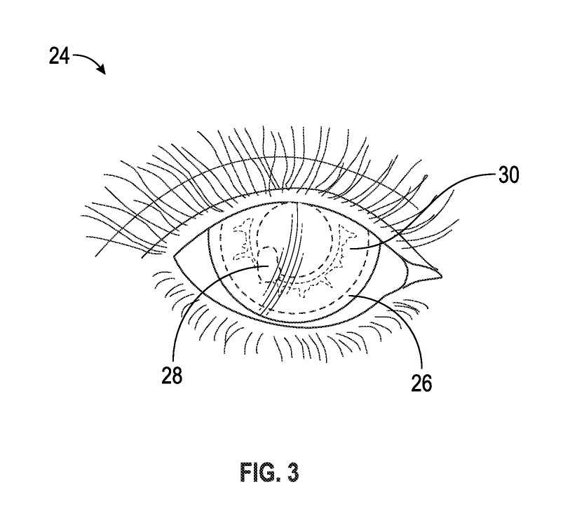

FIG. 3 illustrates a drug delivery system (24) of the present invention for treating ocular diseases in a wide variety of animals at all stages in life (e.g., domesticated cats, dogs, horses, cattle, sheep, swine, goats, including undomesticated animals). “Treating,” “treatment” or “treated” is meant medically managing an animal with the intention that a prevention, cure, stabilization, or amelioration of the symptoms of the disease will result. The drug delivery system (24) may comprise a therapeutic contact lens (26) approximately 6-30 mm in diameter. The therapeutic contact lens (26) may further have a base curvature of approximately 4.49-10.00 mm and a thickness of approximately 10 nm-3 mm.

Collagen.

The therapeutic contact lens (26) may comprise collagen manufactured from porcine or bovine scleral collagen, transgenic tobacco plants, catgut, amongst other sources standard in the industry. Collagen is a safe and naturally occurring protein that biodegrades in the eye (10) after naturally occurring enzymes in the tear film cause the collagen to dissolve. Dissolution rates may vary depending in part upon the degree of collagen cross-linking induced at the time of manufacture (e.g., 1-10 days). The use of collagen in therapeutic contact lens (26) accelerates epithelialization in the eye (10) after irritants cause corneal and/or conjunctival abrasions (28). Collagen's innate properties further enhance the healing of persistent epithelial defects, neurotrophic corneal ulcers, and lubricates the eye to serve as an adjunct in dry eye therapy.

Amniotic Membranes.

As an alternative to collagen or to be used in combination thereof, the therapeutic contact lens (26) may comprise amniotic membranes. Amniotic membranes may be derived from the innermost submucosa of the placenta, an area in which the fetus grows and develops within the mother's uterus. Amniotic membranes may be comprised of a single epithelium layer, a thick basement membrane and an avascular stromal matrix. Amniotic membranes may contain specialized proteins such as fibronectin, laminins, proteoglycans, and glycosaminoglycans. Amniotic membranes may comprise collagen types I, III, IV, V and VII, cytokines and proteinase inhibitors to facilitate wound healing. Amniotic membranes may include antimicrobial qualities and active growth factors that stimulate epithelialization in the eye (10). Epidermal growth factors include transforming growth factor beta (“TGF-b”), fibroblast growth factor (“FGF”), and platelet-derived growth factor (“PDGF”). When incorporated into therapeutic contact lenses (26), amniotic membranes reduce inflammation, angiogenesis, fibrosis and scaring in the cornea (12). Thus, innate properties of amniotic membranes enhance epithelial cell migration, reinforce adhesion of basal epithelial cells, promote epithelial differentiation, and prevent epithelial apoptosis in the eye (10). Amniotic membranes utilized in therapeutic contact lenses (26) typically dissolve in vivo within a period of 1-14 days.

Hydrogels.

As a further alternative to collagen and amniotic membranes or to be used in combination thereof, the therapeutic contact lens (26) may comprise hydrogels. Hydrogels may comprise at least two components: a stable crosslinked polymer matrix; and a less stable aqueous component able to exchange oxygen with the surrounding environment (e.g., water). Exemplary materials utilized in hydrogel contact lenses (26) include etafilcon A, vifilcon A, lidofilcon A, polymacon B, vasurfilcon A, silicone, and a tetrapolymer of hydroxymethylmethacrylate, ethylene glycol, dimethylmethacrylate, and methacrylic acid. Other suitable hydrogel materials known to those skilled in the art may also be utilized. The hydrogels may be ionic or non-ionic comprising between 10% and 90% water by weight. Hydrogel therapeutic contact lenses (26) may be insoluble or may be advantageously biodegradable in vivo over a period of time (e.g., 1-10 days). A notable benefit to hydrogel therapeutic contact lenses is that hydrogels enable up to six times more oxygen to pass through to facilitate healing of discoloration and ulceration (28) of the cornea (12) and to help prevent corneal scaring (28).

As shown in FIG. 3, the therapeutic contact lens (26) of the drug delivery system (24) for treating ocular diseases in animals may further comprise therapeutic drug(s) (30). A variety of therapeutic drug(s) (30) and drug precursors may be utilized to treat ocular diseases in animals including corticosteroids, steroids, growth factors, antibiotics, vitamins and anti-inflammatory compounds (e.g., cyclosporin, sirolimus, rapamycin, cyclophilin A, B, or D inhibitors and derivatives thereof, including other anti-inflammatory compounds standard in the industry). With regard to treating IBK in cattle, the therapeutic drug(s) (30) may comprise oxytetracycline, penicillin, streptomycin, tetracycline, gentamycin, Cloxicillin or combinations thereof, including other antibiotics standard in the industry. The therapeutic drug(s) (30) may further comprise antiviral medications (e.g., trifluridine, ganciclovir) and/or anti-fungal medications (e.g., polyenes: natamycin, nystatin, and amphotericin B; azoles (imidazoles and triazoles): ketoconazole, miconazole, fluconazole, itraconazole, econazole, and clotrimazole; fluorinated pyrimidines: flucytosine). Treating IBK in cattle is illustrated for exemplary purposes, as it is contemplated that the drug delivery system (24) of the present invention may be used to treat other ocular diseases in a wide variety of animals at all stages in life (e.g., domesticated cats, dogs, horses, cattle, sheep, swine, goats, including undomesticated animals).

Therapeutic drug(s) (30) may be incorporated into the therapeutic contact lens (24). For example, therapeutic drugs (30) may be passively transferred into a desiccated or partially desiccated therapeutic contact lens (26). Desiccation may be facilitated by exposing the therapeutic contact lens (26) to ambient or humidity controlled air, heat, or gases (e.g., N₂). Thus, desiccation may remove between 5-75% of an aqueous component (e.g., water) of the therapeutic contact lens (26). The desiccated or partially desiccated therapeutic contact lens (26) may then be soaked in an aqueous dilute solution of the therapeutic drug(s) (30) (“medicated solution”) for a period of time between approximately 30 minutes-24 hours to produce a medicated therapeutic contact lens (24). Alternatively, a fully hydrated therapeutic contact lens (26) may be placed in the medicated solution for a period of time between approximately 30 minutes-24 hours to produce a medicated therapeutic contact lens (26). The concentration of therapeutic drug(s) (30) in the medicated solution may comprise between 0.000001-1000 μg/mL, wherein higher concentrations may also be utilized to reduce transferal times. The therapeutic drug(s) (30) may also be transferred to the therapeutic contact lens (26) from a non-aqueous solvent (e.g., dimethyl sulfoxide) which may be at least partially removed and replaced with an aqueous solution prior to use. It is contemplated that other means standard in the industry for producing a medicated therapeutic contact lens (26) may also be utilized by the drug delivery system (10) of the present invention.

The concentration of therapeutic drug(s) (30) in the medicated therapeutic contact lens (26) should be present in a therapeutically effective amount. A “therapeutically effective amount” is meant an amount of a drug sufficient to produce a preventative, healing, curative, stabilizing, or ameliorative effect in the treatment of ocular diseases in animals. For example, the concentration of therapeutic drug(s) (30) in the medicated therapeutic contact lens (26) may be at least 2-10 times less than that of the medicated solution in which it was soaked. Alternatively, the concentration of therapeutic drug(s) (30) in the mediated therapeutic contact lens (26) may be at least 2-10 times more than that of the medicated solution in which it was soaked depending upon the particular drug's and lens' affinity. Other factors may also influence concentration levels, such as the composition of the medicated solution (e.g., ionic strength and pH), time soaked, temperature of the medicated solution, and type of therapeutic drug(s) (30).

As further shown in FIG. 3, the medicated therapeutic contact lens (26) may be placed in the afflicted eye (10) of an animal with an ocular disease. In particular, the medicated therapeutic contact lens (26) may be placed in contact with the cornea (12) and/or conjunctiva (14) of an afflicted eye (10) for a localized application. The therapeutic drug(s) (30) may be released from the medicated therapeutic contact lens (26) via diffusion into the cornea (12) and/or conjunctiva (14) of the eye (10). The rate of release may be constant over time. As the eye (10) produces tear secretions any ancillary therapeutic drug(s) (30) may be flushed away from the site of application and out of the eye (10). The drug delivery system (24) may release approximately 0.001-1000 μg of the therapeutic drug(s) (30) into the afflicted eye (10) for a sustained time period between approximately 2 hours-10 days. The sustained time period over which the drug delivery system (24) releases the therapeutic drug(s) (30) into the cornea (12) and/or conjunctiva (14) of the afflicted eye (10) may depend on the level of treatment desired and/or the amount of therapeutic drug(s) (30) in the therapeutic contact lens (24). The sustained time period of drug delivery of the present invention (24) is preferable to traditional approaches utilizing topical ointments, gels or drops wherein the therapeutic drugs are immediately flushed away with tear secretions. The sustained time period of drug delivery of the present invention (24) avoids potential harmful side effects incurred in traditional approaches utilizing a highly concentrated drug dosage that is repeated frequently. Moreover, the sustained time period of drug delivery of the present invention (24) reduces—or completely eliminates—the need for frequent repeat dosing required in traditional approaches. Thus, the drug delivery system (24) of the present invention reduces unnecessary stress on the afflicted animal and handler/veterinarian.

The drug delivery system (24) of the present invention has additional benefits over traditional approaches for treating ocular diseases in animals. For instance, the therapeutic contact lens (24) may be biodegradable wherein the lens (26) harmlessly dissolves and is washed away from the eye (10) via tear secretions. Dissolution rates of the biodegradable therapeutic contact lens (26) may range between approximately 2 hours-10 days depending on the level of treatment required. Thus, the therapeutic contact lens (26) may be conveniently left in the eye (10) after treatment of the ocular disease without risk of harm to the animal. Another added benefit of the drug delivery system (24) of the present invention is that it provides ocular surface protection to the eye (10) during treatment. A basic necessity for the eye (10) to successfully heal from an ocular disease or trauma is that it must be protected from eye irritations and the blinking action of the eyelids. The therapeutic contact lens (26) when placed in the eye (10) provides the necessary protection from eye irritants and the blinking action of the eyelids during treatment. Thus, the drug delivery system (24) of the present invention may act as a replacement to traditional approaches utilizing an eye patch or tarsorrhaphy to protect the eye (10) from light, flies and other irritants during the healing process which requires additional handling and recapture of the animal for removal.

FIGS. 1-4 illustrate another aspect of the present invention comprising a method of treating ocular diseases in animals (32). The method of treating ocular diseases in animals (32) includes identifying an animal or a population of animals (34) (e.g., domesticated cats, dogs, horses, cattle, sheep, swine, goats, including undomesticated animals) with an ocular disease (e.g., conjunctivitis, keratitis, and/or keratoconjunctivitis), particularly, IBK in cattle. The method (32) further comprises providing a drug delivery system (24) for treating ocular diseases in a wide variety of animals at all stages in life (36). The drug delivery system (24) may comprise a therapeutic contact lens (26) approximately 6-30 mm in diameter. The therapeutic contact lens (26) may further have a base curvature of approximately 4.49-10.00 mm and a thickness of approximately 10 nm-3 mm.

Collagen.

The therapeutic contact lens (26) may comprise collagen manufactured from porcine or bovine scleral collagen, transgenic tobacco plants, catgut, amongst other sources standard in the industry. Collagen is a safe and naturally occurring protein that biodegrades in the eye (10) after naturally occurring enzymes in the tear film cause the collagen to dissolve. Dissolution rates may vary depending in part upon the degree of collagen cross-linking induced at the time of manufacture (e.g., 1-10 days). The use of collagen in therapeutic contact lens (26) accelerates epithelialization in the eye (10) after irritants cause corneal and/or conjunctival abrasions (28). Collagen's innate properties further enhance the healing of persistent epithelial defects, neurotrophic corneal ulcers, and lubricates the eye to serve as an adjunct in dry eye therapy.

Amniotic Membranes.

As an alternative to collagen or to be used in combination thereof, the therapeutic contact lens (26) may comprise amniotic membranes. Amniotic membranes may be derived from the innermost submucosa of the placenta, an area in which the fetus grows and develops within the mother's uterus. Amniotic membranes may be comprised of a single epithelium layer, a thick basement membrane and an avascular stromal matrix. Amniotic membranes may contain specialized proteins such as fibronectin, laminins, proteoglycans, and glycosaminoglycans. Amniotic membranes may comprise collagen types I, III, IV, V and VII, cytokines and proteinase inhibitors to facilitate wound healing. Amniotic membranes may include antimicrobial qualities and active growth factors that stimulate epithelialization in the eye (10). Epidermal growth factors include transforming growth factor beta (“TGF-b”), fibroblast growth factor (“FGF”), and platelet-derived growth factor (“PDGF”). When incorporated into therapeutic contact lenses (26), amniotic membranes reduce inflammation, angiogenesis, fibrosis and scaring in the cornea (12). Thus, innate properties of amniotic membranes enhance epithelial cell migration, reinforce adhesion of basal epithelial cells, promote epithelial differentiation, and prevent epithelial apoptosis in the eye (10). Amniotic membranes utilized in therapeutic contact lenses (26) typically dissolve in vivo within a period of 1-14 days.

Hydrogels.

As a further alternative to collagen and amniotic membranes or to be used in combination thereof, the therapeutic contact lens (26) may comprise hydrogels. Hydrogels may comprise at least two components: a stable crosslinked polymer matrix; and a less stable aqueous component able to exchange oxygen with the surrounding environment (e.g., water). Exemplary materials utilized in hydrogel contact lenses (26) include etafilcon A, vifilcon A, lidofilcon A, polymacon B, vasurfilcon A, silicone, and a tetrapolymer of hydroxymethylmethacrylate, ethylene glycol, dimethylmethacrylate, and methacrylic acid. Other suitable hydrogel materials known to those skilled in the art may also be utilized. The hydrogels may be ionic or non-ionic comprising between 10% and 90% water by weight. Hydrogel therapeutic contact lenses (26) may be insoluble or may be advantageously biodegradable in vivo over a period of time (e.g., 1-10 days). A notable benefit to hydrogel therapeutic contact lenses is that hydrogels enable up to six times more oxygen to pass through to facilitate healing of discoloration and ulceration (28) of the cornea (12) and to help prevent corneal scaring (28).

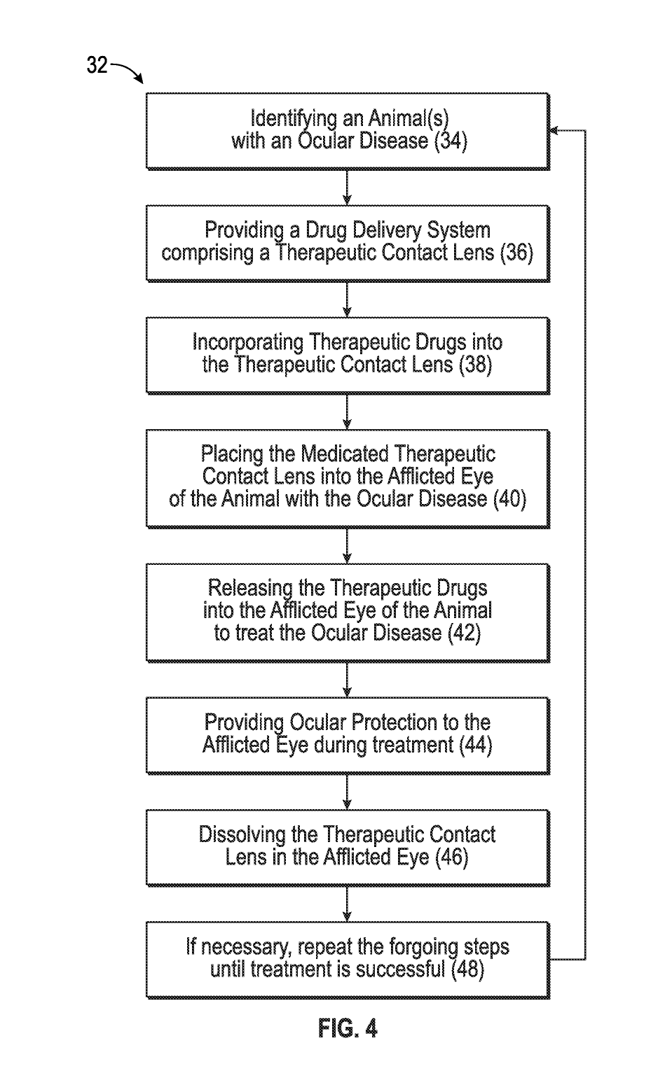

As shown in FIGS. 3-4, the method (32) of the present invention further comprises incorporating (38) therapeutic drug(s) (30) into the therapeutic contact lens (24). For example, therapeutic drugs (30) may be passively transferred into a desiccated (or partially desiccated) therapeutic contact lens (26). Desiccation may be facilitated by exposing the therapeutic contact lens (26) to ambient or humidity controlled air, heat, or gases (e.g., N₂). Thus, desiccation may remove between 5-75% of an aqueous component (e.g., water) of the therapeutic contact lens (26). The desiccated (or partially desiccated) therapeutic contact lens (26) may then be soaked in an aqueous dilute solution of the therapeutic drug(s) (30) (“medicated solution”) for a period of time between approximately 30 minutes-24 hours to produce a medicated therapeutic contact lens (24). Alternatively, a fully hydrated therapeutic contact lens (26) may be placed in the medicated solution for a period of time between approximately 30 minutes-24 hours to produce a medicated therapeutic contact lens (26). The concentration of therapeutic drug(s) (30) in the medicated solution may comprise between 0.000001-1000 μg/mL, wherein higher concentrations may also be utilized to reduce transferal times. The therapeutic drug(s) (30) may also be transferred to the therapeutic contact lens (26) from a non-aqueous solvent (e.g., dimethyl sulfoxide) which may be at least partially removed and replaced with an aqueous solution prior to use. It is contemplated that other means standard in the industry for incorporating therapeutic drug(s) (20) into a therapeutic contact lens (26) may also be utilized by the method of the present invention.

A variety of therapeutic drug(s) (30) and drug precursors may be utilized to treat ocular diseases in animals including corticosteroids, steroids, growth factors, antibiotics, vitamins and anti-inflammatory compounds (e.g., cyclosporin, sirolimus, rapamycin, cyclophilin A, B, or D inhibitors and derivatives thereof, including other anti-inflammatory compounds standard in the industry). With regard to treating IBK in cattle, the therapeutic drug(s) (30) may comprise oxytetracycline, penicillin, streptomycin, tetracycline, gentamycin, Cloxicillin or combinations thereof, including other antibiotics standard in the industry. The therapeutic drug(s) (30) may further comprise antiviral medications (e.g., trifluridine, ganciclovir) and/or anti-fungal medications (e.g., polyenes: natamycin, nystatin, and amphotericin B; azoles (imidazoles and triazoles); ketoconazole, miconazole, fluconazole, itraconazole, econazole, and clotrimazole; fluorinated pyrimidines: flucytosine). Treating IBK in cattle is illustrated for exemplary purposes, as it is contemplated that the drug delivery system (24) of the present invention may be used to treat other ocular diseases in a wide variety of animals at all stages in life (e.g., domesticated cats, dogs, horses, cattle, sheep, swine, goats, including undomesticated animals).

The concentration of therapeutic drug(s) (30) in the medicated therapeutic contact lens (26) should be present in a therapeutically effective amount. A “therapeutically effective amount” is meant an amount of a drug sufficient to produce a preventative, healing, curative, stabilizing, or ameliorative effect in the treatment of ocular diseases in animals. For example, the concentration of therapeutic drug(s) (30) in the medicated therapeutic contact lens (26) may be at least 2-10 times less than that of the medicated solution in which it was soaked. Alternatively, the concentration of therapeutic drug(s) (30) in the mediated therapeutic contact lens (26) may be at least 2-10 times more than that of the medicated solution in which it was soaked depending upon the particular drug's and lens' affinity. Other factors may also influence concentration levels, such as the composition of the medicated solution (e.g., ionic strength and pH), time soaked, temperature of the medicated solution, and type of therapeutic drug(s) (30).

As further shown in FIGS. 3-4, the method (32) of the present invention for treating ocular diseases in animals further comprises placing the medicated therapeutic contact lens (26) in the afflicted eye (10) of an animal (40). In particular, the medicated therapeutic contact lens (26) may be placed in contact with the cornea (12) and/or conjunctiva (14) of an afflicted eye (10) for a localized application. The method further comprises releasing the therapeutic drug(s) (30) from the medicated therapeutic contact lens (26) via diffusion into the cornea (12) and/or conjunctiva (14) of the eye (10) (42). The rate of release may be constant over time. As the eye (10) produces tear secretions any ancillary therapeutic drug(s) (30) may be flushed away from the site of application and out of the eye (10). The drug delivery system (24) may release approximately 0.001-1000 μg of the therapeutic drug(s) (30) into the afflicted eye (10) for a sustained time period between approximately 2 hours-10 days. The sustained time period over which the drug delivery system (24) releases the therapeutic drug(s) (30) into the cornea (12) and/or conjunctiva (14) of the afflicted eye (10) may depend on the level of treatment desired and/or the amount of therapeutic drug(s) (30) in the therapeutic contact lens (24). The sustained time period of drug delivery of the present invention (24) is preferable to traditional approaches utilizing topical ointments, gels or drops wherein the therapeutic drugs are immediately flushed away with tear secretions. The sustained time period of drug delivery of the present invention (24) avoids potential harmful side effects incurred in traditional approaches utilizing a highly concentrated drug dosage that is repeated frequently. Moreover, the sustained time period of drug delivery of the present invention (24) reduces—or completely eliminates—the need for frequent repeat dosing required in traditional approaches. Thus, the method (32) of the present invention for treating ocular diseases in animals reduces unnecessary stress on the afflicted animal and handler/veterinarian.

As shown FIG. 4, the method of the present invention further comprises providing ocular surface protection to the eye (10) during treatment (44). A basic necessity for the eye to successfully heal from an ocular disease or trauma is that the eye (10) must be protected from eye irritations and the blinking action of the eyelids. The therapeutic contact lens (26) when placed in the eye (10) provides the necessary protection from eye irritants and the blinking action of the eyelids during treatment. Thus, the method (32) of the present invention may act as a replacement to traditional approaches utilizing an eye patch or tarsorrhaphy to protect the eye (10) from light, flies and other irritants during the healing process which requires additional handling and recapture of the animal for removal.

As further shown in FIG. 4, the method (32) of the present invention for treating ocular diseases in animals further comprises dissolving the therapeutic contact lens (26) in the eye (10) after treatment (46). Dissolution rates of the biodegradable therapeutic contact lens (26) may range between approximately 2 hours-10 days depending on the level of treatment required. Thus, the therapeutic contact lens (26) may be conveniently left in the eye (10) after treatment of the ocular disease wherein it harmlessly biodegrades and is washed away from the eye (10) via tear secretions. The foregoing steps of the method of the present invention may be repeated if necessary until the ocular disease is successfully treated (48). The method (32) of the present invention for treating ocular diseases in animals may further include traditional methods of isolating the affected animals and utilizing a fly knockdown spray to reduce new pinkeye cases in the overall population.

The drug delivery system (24) of the present invention and method of treating ocular diseases in animals (32) are universally applicable to all animals, particularly cattle, of all sizes, ages and breeds. Furthermore, the drug delivery system (24) of the present invention and method of treating ocular diseases in animals may be used in any geographic location, indoors or outdoors, at any time of year. Although the invention has been described and illustrated with respect to preferred aspects thereof, it is not to be so limited since changes and modifications may be made therein which are within the full intended scope of the invention.

1: A drug delivery system for treating Infectious Bovine Keratoconjunctivitis (“IBK”) in cattle, comprising:
a therapeutic contact lens;
a therapeutic drug for treating IBK;
the therapeutic drug incorporated into the therapeutic contact lens;
the therapeutic contact lens placed on an afflicted eye of the animal with IBK without the aid of a veterinarian;
the therapeutic contact lens is configured to act as a bandage to promote faster healing, protect the afflicted eye from irritants and flies, and reduce pain for the animal;
the therapeutic drug released from the therapeutic contact lens into the afflicted eye of the animal with IBK;
wherein the therapeutic drug is released from the therapeutic contact lens into the afflicted eye of the animal with IBK for a sustained period of time;
wherein the therapeutic drug released from the therapeutic contact lens into the afflicted eye treats the IBK in the animal within 10 days;
wherein a single dosage of the drug delivery system treats IBK in the afflicted eye of the animal;
wherein follow-up doses are eliminated by the drug delivery system;
wherein the therapeutic contact lens is left in the afflicted eye during and after treatment of the IBK.

2: The drug delivery system of claim 1, further comprising:
the therapeutic contact lens is biodegradable;
wherein the biodegradable therapeutic contact lens harmlessly dissolves in the afflicted eye of the animal after treatment and is washed away.

3: The drug delivery system of claim 2, further comprising:
the therapeutic contact lens provides ocular surface protection to the afflicted eye during treatment;
wherein the ocular surface protection guards the afflicted eye from environmental triggers that cause eye irritations.

4: The drug delivery system of claim 1, wherein the therapeutic contact lens comprises:
a) collagen;
b) amniotic membrane(s);
c) hydrogels; or
d) combinations thereof.

5: The drug delivery system of claim 3, wherein the therapeutic contact lens comprises:
a) a diameter of 6-30 mm;
b) a peripheral curvature of 18-32 mm;
c) a base curvature of 12-16 mm; and
d) a thickness of 10 nm-3 mm.

6: The drug delivery system of claim 5, wherein the therapeutic contact lens releases 0.001-1000 μg of the therapeutic drug into the afflicted eye for a sustained time period between 2 hours-10 days.

7: The drug delivery system of claim 6, wherein an edge of the therapeutic contact lens tapers to a point to prevent the animal from removing the lens during treatment of the IBK.

8. (canceled)

9: A method of treating Infectious Bovine Keratoconjunctivitis (“IBK”) in cattle, comprising:
identifying an animal with IBK in a cattle herd;
catching and restraining the animal with IBK;
providing a drug delivery system for treating IBK in cattle with a single dosage, the drug delivery system comprising:
a) a therapeutic contact lens;
b) a therapeutic drug for treating IBK;
c) the therapeutic drug incorporated into the therapeutic contact lens;
d) the therapeutic contact lens is biodegradable;
e) the therapeutic contact lens' innate properties stimulate epithelialization in an afflicted eye with IBK during treatment and prevent corneal scaring;
f) the therapeutic contact lens configured to act as a bandage to promote faster healing, protect the afflicted eye from irritants and flies, and reduce pain for the animal;
placing the therapeutic contact lens on the afflicted eye of the animal with IBK;
releasing the therapeutic drug from the therapeutic contact lens into the afflicted eye of the animal with IBK;
releasing the animal with IBK back into the cattle herd;
treating IBK in the afflicted eye of the animal within 10 days using the drug delivery system; and
dissolving the biodegradable therapeutic contact lens in the afflicted eye of the animal after treatment;
wherein a single dosage of the drug delivery system treats IBK in the afflicted eye of the animal;
wherein follow-up doses are not required by the drug delivery system.

10: The method of treating IBK in cattle of claim 9, wherein the therapeutic contact lens comprises:
a) collagen;
b) amniotic membrane(s);
c) hydrogels; or
d) combinations thereof.

11: The method of treating IBK in cattle of claim 10, wherein the therapeutic contact lens comprises:
a) a diameter of 6-30 mm;
b) a peripheral curvature of 18-32 mm;
c) a base curvature of 12-16 mm; and
d) a thickness of 10 nm-3 mm.

12: The method of treating IBK in cattle of claim 9, further comprising incorporating the therapeutic drug into the therapeutic contact lens via passive transference.

13: The method of treating IBK in cattle of claim 11, wherein the therapeutic contact lens releases 0.001-1000 μg of the therapeutic drug into the afflicted eye for a sustained time period between 2 hours-10 days.

14: The method of treating IBK in cattle of claim 13, wherein dissolution of the biodegradable therapeutic contact lens ranges between 2 hours-10 days.

15: The method of treating IBK in cattle of claim 9, wherein an edge of the therapeutic contact lens tapers to a point to prevent the animal from removing the lens during treatment of the IBK.

16: The method of treating IBK in cattle of claim 2, wherein the therapeutic drug comprises oxytetracycline, penicillin, streptomycin, tetracycline, gentamicin, cloxacillin or combinations thereof.

17: A method of treating Infectious Bovine Keratoconjunctivitis (“IBK”) in cattle, comprising:
identifying an animal with IBK in a cattle herd;
catching and restraining the animal with IBK;
providing a drug delivery system for treating IBK in cattle with a single dosage, the drug delivery system comprising:
a) a desiccated therapeutic contact lens with 50-75% of an aqueous component removed;
b) the therapeutic contact lens having a diameter of 6-30 mm, a peripheral curvature of 18-32 mm: a base curvature of 12-16 mm, and a thickness of 10 nm-3 mm;
c) the therapeutic contact lens having an edge that tapers to a point to prevent the animal from removing the lens during treatment of the IBK;
d) the therapeutic contact lens comprising collagen, amniotic membrane(s), hydrogels, or combinations thereof;
e) a therapeutic drug for treating IBK;
f) the therapeutic contact lens is biodegradable;
g) the therapeutic contact lens' innate properties stimulate epithelialization in an afflicted eye with IBK during treatment and prevent corneal scaring;
h) the therapeutic contact lens configured to act as a bandage to promote faster healing, protect the afflicted eye from irritants and flies, and reduce pain for the animal;
soaking the desiccated therapeutic contact lens in an aqueous medicated solution of the therapeutic drug for a period of time between 12-24 hours, wherein the concentration of therapeutic drug in the aqueous medicated solution is between 0.000001-1000 μg/mL;
incorporating the therapeutic drug into the therapeutic contact lens via passive transference;
placing the therapeutic contact lens in contact with a cornea and/or conjunctiva of the afflicted eye of the animal with IBK without the aid of a veterinarian;
releasing the therapeutic drug from the therapeutic contact lens into the afflicted eye of the animal with IBK;
releasing the animal with IBK back into the cattle herd;
treating IBK in the afflicted eye of the animal within 10 days using the drug delivery system; and
dissolving the biodegradable therapeutic contact lens in the afflicted eye after treatment;
wherein a single dosage of the drug delivery system treats IBK in the afflicted eye of the animal;
wherein follow-up repeat doses are eliminated by the drug delivery system.

18: The method of treating IBK in cattle of claim 17, wherein the therapeutic contact lens releases 0.001-1000 μg of the therapeutic drug into the afflicted eye for a sustained time period between 2 hours-10 days.

19: The method of treating IBK in cattle of claim 18, wherein dissolution of the biodegradable therapeutic contact lens ranges between 2 hours-10 days.

20: The method of treating IBK in cattle of claim 19, further comprising applying a fly knockdown spray on the cattle with IBK to reduce contagion of the cattle herd.
